(19) United States
(12) Plant Patent Application Publication
(10) Pub. No.: US 2005/0034201 P1
(43) Pub. Date: Feb. 10, 2005

(54) Strawberry variety named 'Evangeline'

(50) FRAGARIA ANANASSA; STRAWBERRY

(76) Inventor: Andrew W. Jamieson, Kentville (CA)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/755,607

(22) Filed: Jan. 12, 2004

Related U.S. Application Data
(63) Continuation of application No. 10/073,447, filed on Feb. 11, 2002, now abandoned.

(51) Int. Cl. A01H 5/00

(52) U.S. Cl. PLT/208

(57) ABSTRACT

This invention relates to a strawberry plant (Fragaria ananassa) named ‘Evangeline’ characterized by its large firm fruit and large flowers. The cultivar exhibits high yield when propagated in the field.

### BACKGROUND OF THE INVENTION

The present invention includes a new and distinct cultivar of Fragaria ananassa known by the varietal name ‘Evangeline’, originally designated as “K93-1”. The new variety resulted from a controlled cross in an ongoing breeding program between the strawberry plants. ‘K88-4’ and ‘NYUS119’. ‘K88-4’ is an unpatented variety developed by the Atlantic Food and Horticulture Research Centre (a facility of Agriculture and Agri-Food Canada) in Kentville and ‘NYUS119’ is an unpatented variety developed by the New York State Agricultural Experiment Station in Geneva. ‘Evangeline’ was discovered. in 1993 as a seedling in a controlled breeding plot near Sheffield Mills, Nova Scotia at the Sheffield Farm, a field-station of the Atlantic Food and Horticulture Research Centre, where it was selected and propagated asexually by stolons at the Atlantic Food and Horticulture Research Centre in Kentville. Asexual propagules from this original source have been produced annually in a greenhouse at the Atlantic Food and Horticulture Research Centre, Kentville, Canada. ‘Evangeline’ has been tested at the Atlantic Food and Horticulture Research Centre (starting in 1994) and also, research centres at Charlottetown, Prince Edward Island, Buctouche, New Brunswick, Fredericton, New Brunswick, and Pynn's Brook, Newfoundland, all of Canada. This propagation and testing has demonstrated that the combination of traits disclosed herein which characterize the new variety are fixed and retained true to type through successive generations of asexual reproduction via stolons.

### DESCRIPTION OF THE DRAWINGS

FIG. 1 shows plant parts of the new variety, typical in size, shape, and color;

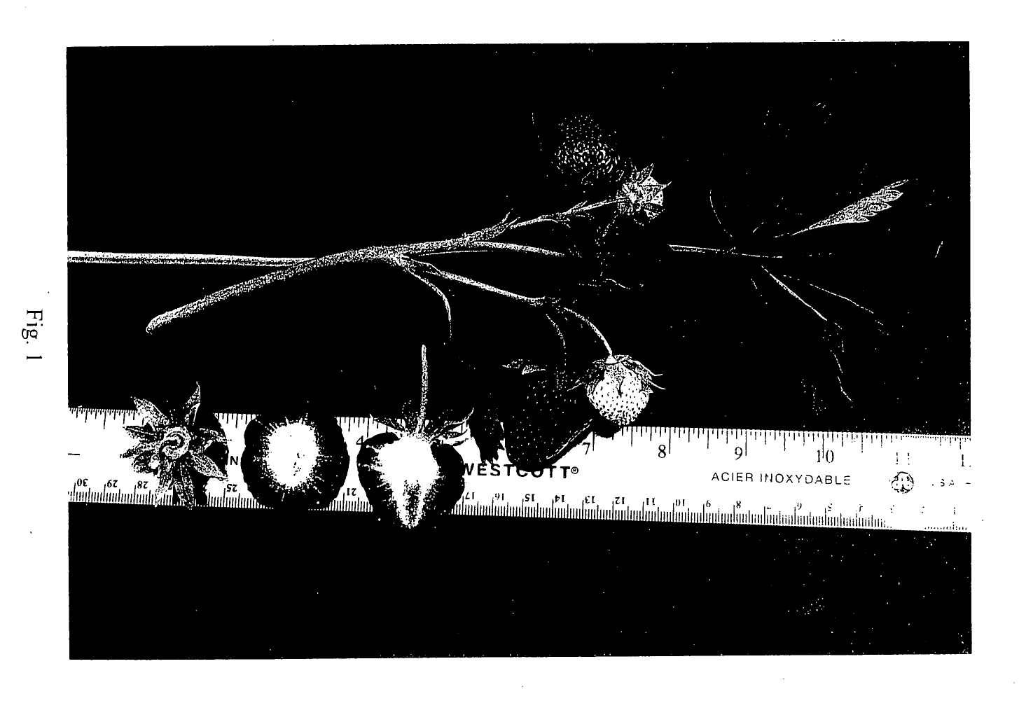

FIG. 2 shows the flowers of the new variety; and

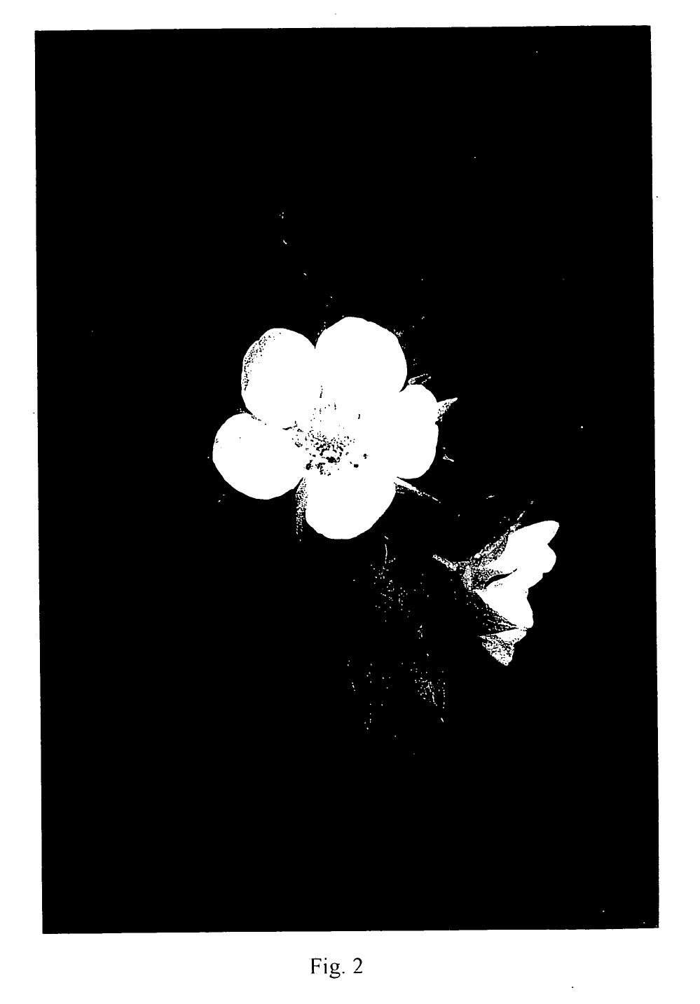

FIG. 3 shows primary fruit of the new variety.

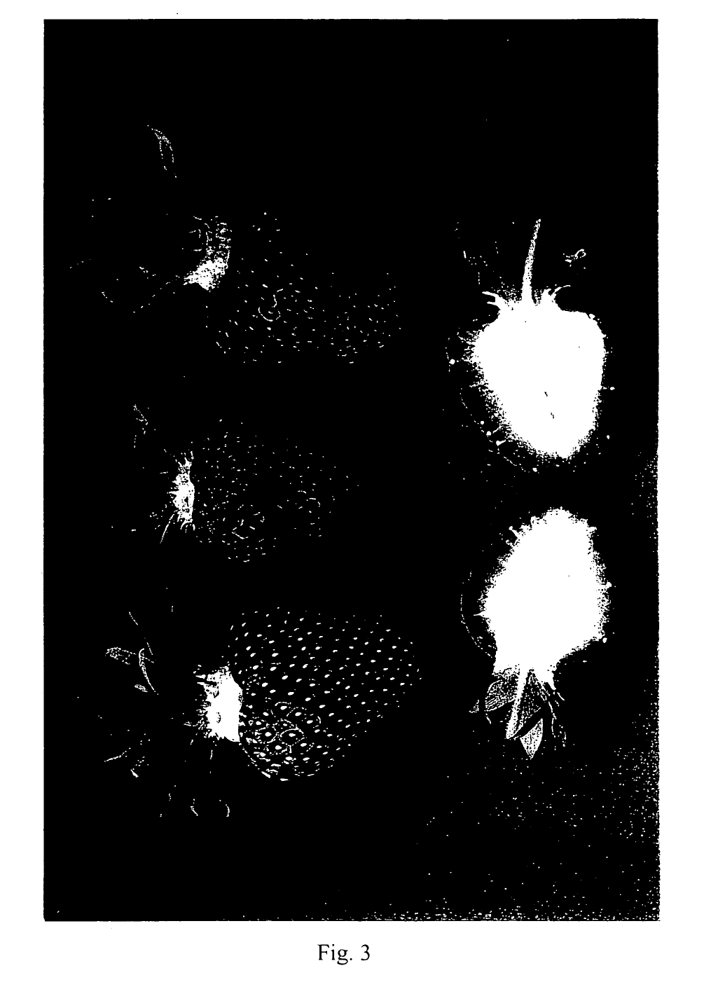

### DESCRIPTION OF THE PLANT

The following detailed botanical description of the new variety is based upon measurements and observations taken of plants and fruit grown in Kentville, Nova Scotia, Canada. Observations were taken from each variety as grown in a side-by-side field trial. Color designations, color descriptions, and other phenotypical descriptions may deviate from the stated values and description depending upon variation in the environment, seasonal, climatic and cultural conditions, however, it is believed that this description will apply to the ‘Evangeline’ plants grown in similar conditions of soil and climate elsewhere. Color references are made to the R.H.S. Colour Chart of the Royal Horticultural Society of London (1966 edition). Descriptive information on the new variety is presented in Tables 1, 2, and 3. In the tables, the flowers described are secondary flowers. The fruit described is the secondary fruit of the maiden crop, thirteen or fourteen months after planting. The harvest data in Table 4 is based on a complete harvest of the crop. The principal differences between ‘Evangeline’ and each of the unpatented variety ‘Veestar’ and ‘Sable’ (patent pending) are set forth.
Classification: The new variety is botanically identified as Fragaria ananassa and commercially classified as a short-day strawberry.

Plant and foliage characteristics: When propagated in the nursery, ‘Evangeline’ runners freely produce similar quantities of runner plants to ‘Veestar’ and ‘Sable’. Individual plants of ‘Evangeline’ are medium to large. The plants of ‘Evangeline’ are of medium density with globose habit and strong vigor. As shown in Table 1, leaf color of ‘Evangeline’, ‘Veestar’ and ‘Sable’ are Green Group.137A on the upper surface and lighter Green Group 137C on the under surface. Leaflets of ‘Evangeline’ and the reference varieties. are about the same size, shape and convexity, and leaves of all three varieties have three leaflets. The leaflet serrations of ‘Evangeline’ are similar in size and number to ‘Veestar’ but for ‘Evangeline’ they are more pointed and the tip serration is smaller than for ‘Veestar’. The venation of ‘Evangeline’ leaflets is pinnate. Petiole pubescence for ‘Evangeline’ tends to be oriented upwards whereas for ‘Veestar’ and ‘Sable’ pubescence is perpendicular.

TABLE 1
Foliar Characteristics for
‘Evangeline’, ‘Veestar’ and ‘Sable’

| | Cultivar | | |
|---|---|---|---|
| Foliar Character | ‘Evangeline’ | ‘Veestar’ | ‘Sable’ |
| Leaf color | Green Group | Green Group | Green Group |
| Upper surface | 137A | 137A | 137A |
| Lower surface | 137C | 137C | 137C |
| Central leaflet | | | |
| Length (mm) | | | |
| Mean | 80.3 | 86.3 | 79.0 |
| Range | 60-93 | 62-110 | 70-93 |
| Width (mm) | | | |
| Mean | 65.7 | 68.0 | 63.9 |
| Range | 47-77 | 50-85 | 55-77 |
| Length/width ratio | 1.22 | 1.27 | 1.24 |
| No. leaflets/leaf | 3 | 3 | 3 |
| Leaf convexity | cupped | cupped | cupped |
| Serrations | | | |
| Number | moderate | moderate | many |
| Size | medium | medium | small |
| Shape | semi-pointed | semi-round | semi-round |
| Tip serration size | small | medium | small |
| Leaf pubescence | medium | medium | medium |
| Petiole pubescence | | | |
| Density | sparse | sparse | sparse |
| Direction | upwards | perpendicular | perpendicular |

Flower and fruit characteristics: The length of bloom for ‘Evangeline’ is about three weeks when grown in Kentville, Nova Scotia, Canada in a matted row cultural system. Flowering for ‘Evangeline’, ‘Veestar’, and ‘Sable’ typically begins on May 21 and ends on June 11. As shown in Table 2, the flower truss of ‘Evangeline’ is shorter than for ‘Sable’ and opens slightly lower in relation to the canopy. ‘Evangeline’ flower trusses produce fewer flowers than do the trusses of ‘Veestar’ and ‘Sable’. Flowers of ‘Evangeline’ and the reference varieties are white. The anther color is Yellow-Orange Group 17A. Secondary flowers of ‘Evangeline’ usually have 5 petals, and ‘Evangeline’ flowers are larger than those of ‘Veestar’ and ‘Sable’. Petals of ‘Evangeline’ are longer than wide in contrast to the petals of ‘Veestar’ and ‘Sable’ which are wider than long. Petals of ‘Veestar’ and ‘Sable’ tend to overlap while those of ‘Evangeline’ are spaced. Trusses of ‘Evangeline’ are erect at first picking and continue to be erect while those of ‘Veestar’ and ‘Sable’ are semi-erect becoming prostrate as harvest progresses. ‘Evangeline’ has a larger calyx than does ‘Veestar’ and ‘Sable’. The position of the calyx is raised for ‘Evangeline’, even with the top of the fruit for ‘Veestar’ and often in a basin for ‘Sable’. The calyx is easily separated from the fruit for all three varieties. The fruit of ‘Evangeline’ are larger than for ‘Veestar’ but both are conic in shape while the fruit of ‘Sable’ are wider than long giving a cordate shape. The seeds of ‘Evangeline’ are more indented than for ‘Veestar’ and ‘Sable’ and ‘Evangeline’ fruit has firmer flesh. The fruit color of ‘Evangeline’ varies between the lighter Red Group 45A of ‘Sable’ to the darker Red Group 46A of ‘Veestar’. The pith of ‘Evangeline’ is lighter than for ‘Veestar’ and ‘Sable’. The achenes of ‘Evangeline’ are Green-Yellow Group 1A but darken to Orange-Red Group 34A on the side of the fruit when exposed to sunlight.

TABLE 2
Flower and Fruit Characteristics for
‘Evangeline’, ‘Veestar’, and ‘Sable’.

| | Cultivar | | |
|---|---|---|---|
| Character | ‘Evangeline’ | ‘Veestar’ | ‘Sable’ |
| Flower position | even or | beneath | even |
| (relative to leaf canopy) | slightly beneath | | |
| Flower truss length | short-medium | short-medium | medium-long |
| | 23.8 | 22.6 | 28.8 cm |
| Number of flowers/truss | 5.4 | 6.9 | 8.0 |
| Number of petals | 5.0 | 5.5 | 5.3 |
| Flower size (mm | 27.9 | 23.6 | 25.2 |
| diameter) | | | |
| Flower color | White | White | White |
| Petal length (mm) | 11.6 | 9.2 | 9.6 |
| Petal width (mm) | 10.4 | 10.3 | 11.4 |
| Petal spacing | spaced | slightly | slightly |
| | | overlapping | overlapping |
| Calyx size | | | |
| Inner calyx (mm diam.) | 27.7 | 21.2 | 24.6 |
| Outer calyx (mm diam.) | 28.1 | 19.7 | 24.2 |
| Calyx position | raised | even | even to in a |
| | | | basin |
| Adherence of the calyx | weak | weak | weak |
| Fruit size and shape | | | |
| Length (mm) | 30.3 | 27.5 | 25.1 |
| Width (mm) | 29.1 | 25.5 | 32.0 |
| Length/width ratio | 1.04 | 1.08 | 0.78 |
| Subjective fruit shape | conic to | conic to | cordate |
| | long-conic | short-conic | |
| Seed position | indent | even | slight indent |
| Fruit firmness | firm | soft | medium |
| Fruit firmness (N) | 3.4 | 2.8 | 3.0 |
| Skin toughness (g) | 10.3 | 8.4 | 11.6 |
| Color (R.H.S. Colour | | | |
| Chart) | | | |
| Calyx | Green Group | Green Group | Green Group |
| | 137C | 137C | 137C |
| Fruit exterior | Red Group | Red Group | Red Group |
| | 45A to 46A | near 46A | 45A |
| Fruit interior | | | |
| Pith | Red Group | Red Group | Red Group |
| | 41A | near 46B | 42B |
| Cortex | Red Group | Red Group | Red Group |
| | 45B | near 46B | 42A |

Disease resistance: ‘Evangeline’ and ‘Veestar’ have a similar pattern of resistance to red stele root rot (Phytophthora fragariae) and they show much less resistance than ‘Sable’ which is resistant to the prevalent A-6 race, as shown in Table 3. ‘Evangeline’ is resistant to powdery mildew (Sphaerotheca macularis) while ‘Veestar is moderately resistant and ‘Sable’ is susceptible. ‘Evangeline’ and ‘Veestar’ are less affected by fruit rot (Botrytis cinerea) than ‘Sable’.

TABLE 3
Resistance of ‘Evangeline’, ‘Veestar’, and ‘Sable’ to
races of Phytophthora fragariae (red stele root rot)

| | | Cultivar | | |
|---|---|---|---|---|
| Canadian race | American race | ‘Evangeline’ | ‘Veestar’ | ‘Sable’ |
| C-1 | | S | S | I |
| C-2 | A-6 | S | S | R |
| C-3 | A-4 | R | R | R |
| C-4 | A-3 | I | S | R |
| C-5 | A-5 | S | S | S |
| C-6 | A-7 | R | R | S |
| C-7 | | S | S | S |
| C-8 | | S | S | S |

S = Susceptible;
I = Intermediate;
R = Resistant

Production characteristics: ‘Evangeline’ has been widely tested for several years. ‘Evangeline’ has produced moderate yields, generally similar to ‘Meestar’ but below ‘Sable’, as shown in Table 4. The percent marketability of ‘Evangeline’ is generally above the marketability of ‘Veestar’ and ‘Sable’. The fruit of ‘Evangeline’ are much larger (by weight) than ‘Veestar’ but similar to ‘Sable’. The mean harvest date for ‘Evangeline’ is about the same as ‘Veestar’ or about one day later. Subjectively, the flavor of the fruit of ‘Evangeline’ is rated good to very good when fully ripe.

TABLE 4
Performance of ‘Evangeline’ and Veestar’ or ‘Sable’ for
1997, 1998 and 2000 averaged over several sites.*

| | Total Yield | % Yield | Size | Mean Harvest |
|---|---|---|---|---|
| | (t/ha) | Marketable | (g/fruit) | (day of year) |
| 1997 (five sites) | | | | |
| ‘Evangeline’ | 4.2 | 91.7 | 10.1 | 197.6 |
| ‘Veestar’ | 6.4 | 82.6 | 8.3 | 196.8 |
| ‘Sable’ | 7.4 | 87.7 | 10.7 | 197.7 |
| 1998 (four sites) | | | | |
| ‘Evangeline’ | 5.06 | 80.1 | 9.2 | 186.0 |
| ‘Veestar’ | 6.38 | 71.6 | 9.4 | 185.4 |
| ‘Sable’ | 7.52 | 72.8 | 9.7 | 185.5 |
| 1998 (one site) | | | | |
| ‘Evangeline’ | 11.61 | 96.8 | 13.2 | 179.2 |
| ‘Veestar’ | 12.04 | 92.4 | 8.0 | 178.3 |
| ‘Sable’ | 19.74 | 87.1 | 11.7 | 180.3 |
| 2000 (three sites) | | | | |
| ‘Evangeline’ | 6.0 | 93.1 | 9.7 | 192.4 |
| ‘Sable’ | 11.5 | 87.1 | 9.6 | 191.7 |

*Kentville NS, Charlottetown PEI, Fredericton NB, Buctouche NB, and Pynn's Brook Nfld. Plants were grown in matted rows and three blocks of 3 m long rows were harvested at each site.

1. The new and distinct variety of strawberry plant named ‘Evangeline’ as described and illustrated.
